(19) United States
(12) Patent Application Publication
(10) Pub. No.: US 2003/0228373 A1
(43) Pub. Date: Dec. 11, 2003

(54) Composition including a triamine and a biocide and a method for inhibiting the growth of microorganisms with the same

(75) Inventors: Michael Ludensky, Randolph, NJ (US); Christopher Hill, Wanaque, NJ (US); Florian Christof Albrecht Lichtenberg, Grenzach (DE)

Correspondence Address: DARBY & DARBY P.C., P. O. BOX 5257, NEW YORK, NY, 10150-5257, US

(73) Assignees: Lonza Inc.; Lonza AG

(21) Appl. No.: 10/349,151

(22) Filed: Jan. 22, 2003

Related U.S. Application Data
(60) Provisional application No. 60/350,677, filed on Jan. 22, 2002.

(51) Int. Cl. A61K 31/66; A61K 31/53; A61K 31/19; A61K 33/40; A01N 39/00; A01N 33/02

(52) U.S. Cl. 424/600; 424/661; 514/241; 424/616; 424/640; 514/75; 514/557; 514/674

(57) ABSTRACT

The present invention is directed to a composition for inhibiting microbial growth, which includes a biocide (other than a triamine) and a triamine. The biocide is an oxidizing biocide, a non-oxidizing biocide, or a combination thereof. This composition is particularly effective at preventing the growth of various microorganisms in aqueous solutions, such as industrial waters. The present invention also relates to a method of controlling the growth of microorganisms in an aqueous composition by adding the composition of the present invention to the solution.

This application claims the benefit of U.S. Provisional Application No. 60/350,677, filed on Jan. 22, 2002, which is hereby incorporated by reference.

### FIELD OF THE INVENTION

The present invention relates to compositions containing a triamine and a biocide and methods for inhibiting microbial growth in water with the same.

### BACKGROUND OF THE INVENTION

Control of microbial growth in industrial waters is a major concern for industrial manufacturers. Microbial deposits in industrial waters causes corrosion and breakage of machinery, which results in loss of production due to increased down time. Bacteria, such as Thiobacillus, also cause breakage of concrete structures, for example at waste treatment facilities, by oxidation. Furthermore, microorgansims may result in decreased yield and loss of product purity. Microbial growth is particularly troublesome in paper mill industries. Growth of microbial slime deposits and biofilms in pulp and paper mill waters and papermaking slurries have been estimated to be responsible for nearly 70% of all machinery breakages, blockages, and pump failures.

A biofilm forms when bacteria adhere to surfaces in aqueous environments and begin to excrete a slimy, glue-like substance that can anchor them to any kind of material (e.g., metal or plastic). A biofilm can be formed by a single bacterial species, but more often biofilms consist of many species of bacteria, as well as fungi, algae, protozoa, debris, and corrosion products. Essentially, biofilm may form on any surface exposed to bacteria and water. Bioflims frequently interfere in industrial processes involving cooling water towers, mining process waters, and food processing waters.

Conventionally, microbial growth in industrial waters is controlled with oxidizing and non-oxidizing biocides. Although many of these biocides effectively inhibit microbial growth, their effectiveness is short-lived. Exposure to harsh physical or chemical conditions found in industrial waters rapidly reduces the efficacy of the biocides. As a result, the concentration of biocide added to the industrial water must be substantially greater than the minimum necessary to inhibit the microbial growth in pure water. Since many biocides are expensive, this approach is often not economically feasible. Furthermore, many biocides are highly toxic and regulated by the government. This issue is particularly relevant at paper production facilities, which use large quantities of industrial water in manufacturing processes.

European Patent No. 333,143-B1 discloses liquid detergents containing N,N-bis-(3-aminopropyl)lauryl amine as a biocidally active substance.

The present invention provides a convenient, economically advantageous solution to the problem of controlling microbial growth in industrial water. The compositions of the present invention include a combination of substances, which have microbicidally synergistic effects. The synergistic effect reduces the need to use large quantities of biocides in industrial waters to control microbial growth.

### SUMMARY OF THE INVENTION

The present invention is directed to a composition for inhibiting microbial growth, which includes a biocide (other than a triamine) and a triamine. The biocide is an oxidizing biocide, a non-oxidizing biocide, or a combination thereof. Suitable triamines include those having the formula

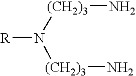

where R is a substituted or unsubstituted C₈ to C₁₈ alkyl, C₈ to C₁₈ alkenyl, C₈ to C₁₈ alkynyl, C₈ to C₁₈ cycloalkyl or aryl, and R is optionally interrupted by one or more heteroatoms. This composition is particularly effective at preventing the growth of various microorganisms in aqueous solutions, such as industrial waters.

The present invention also relates to a method of controlling the growth of microorganisms in an aqueous composition, by adding the composition of the present invention to the solution.

### DETAILED DESCRIPTION OF THE INVENTION

An oxidizing biocide is a chemical agent that has a microbial effect due to its strong oxidizing power, which is directed unspecifically towards organic matter including microorganisms.

A non-oxidizing biocide is a chemical agent that has a microbial effect due to its chemical properties other than its oxidizing power, which is directed specifically or non-specifically towards microorganisms.

Suitable oxidizing biocides include, but are not limited to, peracetic acid, ozone, sodium hypochlorite, calcium hypochlorite, lithium hypochlorite, chlorine dioxide, bromochlorodimethylhydrantoin, dichloromethylethylhydrantoin, dichlorodimethylhydrantoin, trichlorocyanuric acid, sodium dichlorocyanuric acid, stabilized bromine, bromine chloride, brominated sulfamic acid, chlorinated sulfamic acid, sodium percarbonate, potassium monopersulfate, magnesium monoperphthalate, potassium permanganate, hydrogen peroxide, and combinations thereof.

The oxidizing biocide peracetic acid may be formed in situ, such as in water used in wood, pulp, or paper making processes. Therefore, the composition of the present invention may include the triamine, a peracetic acid precursor, and, optionally, a peracetic acid generator. A non-limiting example of a peracetic acid precursor is tetraacetylethylene diamine (TAED). The reaction of TAED with a peracetic acid generator, such as a peracid (e.g. hydrogen peroxide or sodium perborate), yields peracetic acid. Preferably, the composition includes a sufficient amount of triamine, peracetic acid precursor, and, optionally, peracetic acid generator to generate a synergistic microbiocidally effective amount of triamine and peracetic acid. Preferably, compositions containing the peracetic acid generator are in solid form, thereby preventing them from generating peracetic acid until added to an aqueous solution. A preferred composition includes N,N-bis(3-aminopropyl)-dodecylamine, a peracetic acid precursor (preferably TAED), and optionally, a peracetic acid generator. In wood, pulp, and paper making applications, a composition comprising N,N-bis(3-aminopropyl)-dodecylamine and the peracetic acid precursor may be added to process waters containing a peracid (e.g., hydrogen peroxide) to yield a mixture containing N,N-bis(3-aminopropyl)-dodecylamine and peracetic acid. Alternatively, the composition containing N,N-bis(3-aminopropyl)-dodecylamine and the peracetic acid precursor may also include the peracetic acid generator in, preferably, solid form.

Suitable non-oxidizing biocides include, but are not limited to, dibromonitrilopropionamide (DBNPA), decylthioethanamine (DTEA), quaternary ammonium compounds, that include, but are not limited to alkyl dimethyl benzyl ammonium chloride, dialkyl dimethyl ammonium chloride, didecyl dimethyl ammonium chloride, dioctyl dimethyl ammonium chloride, octyl decyl dimethyl ammonium chloride, alkyl dimethyl ethyl benzyl ammonium chloride, diisobutylphenoxyethoxyethyl dimethyl benzyl ammonium chloride, and didecylmethylpolyoxyethylammonium propionate, polymeric quaternary ammonium compounds (polyquats), that include, but are not limited to poly[oxyethylene(dimethyliminio)ethylene(dimethyliminio)ethylene dichloride], poly[hydroxyethylene(dimethyliminio)ethylene(dimethyliminio)methylene dichloride], poly[hydroxyethylene(dimethyliminio)-2-hydroxypropylene(dimethyliminio)methylene dichloride, [N-[3-(dimethylammonio)propyl]-N′[3-(ethyleneoxyethylenedimethylammonio)propyl]urea dichloride], dodecyl guanidine hydrochloride (DGH), β-bromo-β-nitrostyrene (BNS), tetrakishydroxymethylphosphonium sulfate (THPS), bis(trichloromethyl)sulfone (BTS) and mixtures thereof.

Any non-oxidizing or oxidizing biocidal compound, which partially or fully inhibits growth of any microorganism is within the scope of the present invention. Suitable non-oxidizing and oxidizing biocides are obtainable from several chemical suppliers, including Lonza Inc. of Fair Lawn, N.J.

Suitable triamine compounds include, but are not limited to, those having the formula

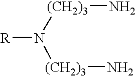

where R is a substituted or unsubstituted C₈ to C₁₈ alkyl, C₈ to C₁₈ alkenyl, C₈ to C₁₈ alkynyl, or C₈ to C₁₈ cycloalkyl or aryl, and R is optionally interrupted with one or more heteroatoms.

The terms “alkyl”, “alkenyl”, and “alkynyl” as used herein include straight and branched alkyl, alkenyl, and alkynyl substituents. An “alkyl” group is a saturated hydrocarbon. An “alkenyl” group is an unsaturated hydrocarbon having one or more double bonds. An “alkynyl” group is an unsaturated hydrocarbon having one or more triple bonds.

Non-limiting examples of alkyl groups which are within the scope of the invention include methyl-, ethyl-, propyl-, butyl-, pentyl-, hexyl-, heptyl-, octyl-, nonyl-, decyl-, undecyl-, dodecyl-, tridecyl-, tetradecyl-, pentadecyl-, hexadecyl-, heptadecyl-, and octadecyl-groups. Also, alkenyl and alkynyl versions of all these alkyl groups where one or more double bonds or triple bonds, respectively, are present in the groups are within the scope of the invention.

A “cycloalkyl” is a cyclic, saturated hydrocarbon group. It may contain fused aliphatic rings, i.e., aliphatic rings that share a common carbon-carbon bond. R may also include bridged aliphatic rings, i.e. rings that are not fused but connected by another alkyl, alkenyl, alkynyl or heteroatomic group. Examples of cycloalkyl groups which are within the scope of the invention include, but are not limited to cyclopropyl-cyclobutyl-, cyclopentyl-, cyclohexyl-, cycloheptyl-, cyclooctyl-, cyclononyl-, cyclodecyl-, cycloundecyl-, cyclododecyl-, cyclotridecyl-, cyclotetradecyl-, cyclopentadecyl-, cyclohexadecyl-, cycloheptadecyl-, cyclooctadecyl-groups. Cycloalkyl groups may be interrupted by one or more heteroatoms including O, N, P or S. Examples of interrupted cycloalkyl groups include, but are not limited to, tetrahydrofuranyl, tetrahydropyranyl, piperidinyl, morpholino, and pyrrolindinyl groups.

An “aryl” is a cyclic aromatic moiety. Aromatic moieties are unsaturated cyclic hydrocarbons containing one or more rings, typified by benzene. Aryl groups may contain fused rings, e.g., an aromatic ring that shares a common carbon-carbon bond with an aliphatic ring (e.g., a cycloalkyl or a heterocycloalkyl) or with another aromatic ring, i.e., an aryl. Typical fused aryl groups include, but are not limited to, naphathalenyl-, indenyl-, and pentalenyl groups. Aryl groups may also be interrupted by one or more heteroatoms including O, N, P or S. Some examples of interrupted aryl groups include, but are not limited to, pyrolyl-, imidazolyl-, pyrazolyl-, pyridinyl-, pyrazinyl-, pyridazinyl-, isoindolyl-, furyl-, pyranyl-, isobenzofuranyl-, thienyl-, benzothienyl, chromenyl-, indoly-, and quinolyl-groups.

The term “substituted” as used herein includes compounds substituted with one or more of halogen (such as F, Cl, I, or Br); heteroatomic groups; C₈ to C₁₈ alkyl; C₈ to C₁₈ alkenyl; C₈ to C₁₈ alkynyl; C₈ to C₁₈ cycloalkyl; aryl; or carbonyl containing C₈ to C₁₈ alkyl, C₈ to C₁₈ alkenyl, or C₈ to C₁₈ alkynyl groups (such as ketones, esters, ethers, carbonates, or carboxylates).

Suitable heteroatoms include, but are not limited to, O, N, P, and S.

Suitable heteroatomic groups include, but are not limited to, —NH₂, —NO₂, —SO₂, —SO₃, —PO₃, ═O, and —OH.

The substituent R is preferably unsubstituted C₈ to C₁₈ alkyl, C₈ to C₁₈ alkenyl, C₈ to C₁₈ alkynyl, C₈ to C₁₈ cycloalkyl, or aryl. Preferred triamines include, but are not limited to, N,N-bis(3-aminopropyl)-dodecylamine, available as Lonzabac® 12 from Lonza Inc. of Fair Lawn, N.J., bis(3-aminopropyl)octylamine, and N,N-bis(3-aminopropyl)-octylamine.

The composition broadly includes a synergistically microbicidal effective amount of the triamine and biocide. In a preferred embodiment, the biocide is present in the composition at from about 1 to about 500 parts per million (ppm). The concentration of biocide can be as low as 0.0001, 0.001, 0.01, 0.1, or 0.5 ppm and as high as 1,000, 5,000, 10,000, or 100,000 ppm. Furthermore, concentrations of biocide within the range of from about 50 to about 450 ppm, from about 100 to about 400 ppm, from about 150 to about 350 ppm, or from about 200 to about 300 ppm are within the scope of the present invention.

In another preferred embodiment, the triamine is present in the composition at from about 1 to about 500 parts per million (ppm). The concentration of triamine can be as low as 0.0001, 0.001, 0.01, 0.1, or 0.5 ppm and as high as 1,000, 5,000, 10,000, or 100,000 ppm. Furthermore, concentrations of triamine within the range of from about 50 to about 450 ppm, from about 100 to about 400 ppm, from about 150 to about 350 ppm, or from about 200 to about 300 ppm are within the scope of the present invention.

The weight ratio of biocide to triamine preferably ranges from about 1:500 to about 500:1 and more preferably ranges from about 1:250 to about 250:1. The weight ratio can be as low as 1:1000, 1:5000, 1:10000, 1:50000, or 1:100000 and as high as 1000:1, 5000:1, 10000:1, 50000:1, or 100000:1.

A preferred composition includes N,N-bis(3-aminopropyl)dodecylamine and peracetic acid. According to a preferred embodiment, the composition includes a synergistic microbiocidally effective amount of N,N-bis(3-aminopropyl)dodecylamine and peracetic acid. More preferably, the composition includes from about 30 to about 1,000 ppm of N,N-bis(3-aminopropyl)dodecylamine and from about 20 to about 1,000 ppm of peracetic acid. Even more preferably, the composition includes from about 30 to about 200 ppm of N,N-bis(3-aminopropyl)-dodecylamine and from about 20 to about 200 ppm of peracetic acid.

Another preferred composition includes N,N-bis(3-aminopropyl)dodecylamine and N-(C₁₂-C₁₆ alkyl)-N-benzyl-N,N-dimethylammonium chloride. According to a preferred embodiment, the composition includes a synergistic microbiocidally effective amount of N,N-bis(3-aminopropyl)-dodecylamine and N-(C₁₂-C₁₆ alkyl)-N-benzyl-N,N-dimethylammonium chloride. More preferably, the composition includes from about 30 to about 1,000 ppm of N,N-bis(3-aminopropyl)dodecylamine and from about 20 to about 1,000 ppm of N-(C₁₂-C₁₆ alkyl)-N-benzyl-N,N-dimethylammonium chloride. Even more preferably, the composition includes from about 30 to about 200 ppm of N,N-bis(3-aminopropyl)dodecylamine and from about 20 to about 200 ppm of N-(C₁₂-C₁₆ alkyl)-N-benzyl-N,N-dimethylammonium chloride.

The composition may include one or more solvents, such as water, deionized water, butildiglycol, propylenglycol, and alcohols such as ethanol or 1-propanol. Preferred solvents include one or more of water, butildiglycol, and propylenglycol.

The composition may also include one or more adjuvants, such as thickeners, buffers, colorants, fragrances, surfactants, builders, complexing agents such as EDTA, pH regulators, defoamers, foam stablizers, and corrosion inhibitors.

The composition of the present invention controls the growth of microorganisms, kills microorganisms, or both. The growth of bacteria, fungi, molds, algae and protozoa, may be inhibited with the composition of the present invention. In particular, the composition may inhibit the growth of coliform bacteria, such as Escherichia coli, Enterobacter aerogenes and Klebsiella pneumoniae; Legionella bacteria; protozoa of the Giardia genus, such as Giardia lamblia and Cryptosporidium; the fungus Candida albicans; and mold, such as the mold myxomycota. The composition may also inhibit the growth of biofilm forming microbes, such as Pseudomonas aeruginosa, Staphylococcus aureus, Shewanella, and Aeromonas.

The compositions of the invention can inhibit microbial growth in any system, including aqueous solutions and systems, such as industrial waters. The term “industrial waters” refers to any body of water created by or used in industrial processes or existing in any machine or device. Non-limiting examples of industrial waters include condensed water in air conditioning systems, water used in wood, pulp, or paper making processes, cooling water, and food or beverage process water. The composition of the present invention is particularly useful for inhibiting the growth of Legionella bacteria in industrial waters present in air conditioning systems.

The aqueous solution can be (i) a circulating water slurry comprising organic matter or (ii) a slurry dilution water. Generally, a slurry dilution water contains little (<0.2% by weight), if any, organic matter. Slurry dilution waters are frequently added to dilute or form solutions containing organic matter, especially pulp. Furthermore, slurry dilution water is frequently recovered from circulating water slurries containing organic matter by methods known in the art. The solution may be, for example, a pulp slurry, a papermaking slurry, a mineral slurry or white water. White water is generally separated liquid that is re-circulated to a preceding stage of a papermaking process, especially to the first disintegration stage, where paper, water and chemicals are mixed.

Generally, a mineral slurry comprises of from about 50 to about 80% by weight of mineral matter, such as, but not limited to, calcium carbonate or clay. The mineral slurry may also contain an organic dispersing agent. Preferred organic dispersing agents include, but are not limited to, polyacrylates.

Typical pulp slurries in paper applications contain from about 0.2 to about 18% by weight of organic matter, based upon 100% total weight of slurry. The organic matter is typically comprised of wood fiber (or pulp) and adjuvants, such as sizing and starch. Generally, the organic matter comprises from about 90 to about 99% by weight of wood fiber (or pulp), based upon 100% total weight of organic matter. According to a preferred embodiment, the wood fiber is at least partially derived from recycled paper.

The pulp slurry may also contain other adjuvants known in the art. Examples of such adjuvants include, but are not limited to, slimicides; sodium hydroxide (or other caustic); peroxide stabilizers, such as sodium silicate, magnesium sulfate, and polyphosphates; chelating agents, such as EDTA; fatty acids; and combinations thereof.

Generally, the pH of the solution ranges from about 7 to about 13 and preferably from about 8 to about 11. In another embodiment, the pH of the solution ranges from about 4 to about 13, preferably from about 7 to about 12, and more preferably from about 8 to about 11.

The following examples are intended to describe the present invention without limitation. All percentages are by weight unless otherwise indicated.

### EXAMPLE 1

Samples of process water having a pH of 7.0 obtained from a paper mill were supplemented with Lonzabac® 12.100 and Barquat® MB-80 at the concentrations indicated in Table 1 below. Barquat® MB-80 is an 80% aqueous solution of N-(C₁₂-C₁₆ alkyl)-N-benzyl-N,N-dimethylammonium chloride available from Lonza Inc. of Fair Lawn, N.J. Lonzabac® 12.100 is a 100% N,N-bis(3-aminopropyl)-dodecylamine liquid available from Lonza Inc. The samples were immediately mixed and incubated for 3 hours at 37° C. with agitation in an orbital shaker. After 3 hours, tryptone glucose extract (TGE) agar plates were inoculated with each sample and grown overnight at 37° C. The number of colonies on each plate was then counted. The results are shown in Table 1.

This experiment was repeated with a sample of process water that was not treated with Lonzabac® 12.100 or Barquat® MB-80 as a control.

TABLE 1

| | | | Logarithmic Growth |
|---|---|---|---|
| | | | Reduction relative to the |
| | | Colony forming units per | Control Experiment |
| | | milliliter of sample | [log (CFU/ml of control |
| Lonzabac ® 12.100 | Barquat ® MB-80 | (CFU/ml) after overnight | plate) − log (CFU/ml of |
| (ppm) | (ppm) | incubation | experimental plate)] |
| 15 | 0 | 6.55 × 10⁷ | 0.7 |
| 30 | 0 | 6.40 × 10⁶ | 1.7 |
| 60 | 0 | 3.25 × 10⁵ | 3.0 |
| 90 | 0 | 1.00 × 10⁵ | 3.5 |
| 0 | 20 | 1.72 × 10⁷ | 1.3 |
| 0 | 50 | 2.2 × 10⁵ | 3.2 |
| 15 | 10 | 6.4 × 10⁶ | 1.7 |
| 15 | 20 | 4.3 × 10⁶ | 1.9 |
| 30 | 10 | 2.55 × 10⁶ | 2.1 |
| 30 | 20 | 2.3 × 10⁵ | 3.1 |

| | | |
|---|---|---|
| Control | 3.16 × 10⁸ | — |

Synergism for the solutions in Table 1 against the microorganisms in the process water was calculated by the methods described in C. E. Kull et al., “Mixtures of Quaternary Ammonium Compounds and Long-chain Fatty Acids as Antifungal Agents”, Applied Microbiology, 9:538-541 (1961). The synergism value (Q_{A}/Qₐ+Q_{B}/Q_{b}) was determined. Q_{A} is the concentration of Lonzabac® 12.100 (in ppm) in a Lonzabac® 12.100/Barquat® MB-80 mixture, which yielded a thousand fold reduction in the microorganism count of the process water. Qₐ is the concentration of Lonzabac® 12.100 alone (in ppm) required to yield a thousand fold reduction in the microorganism count of the process water. Q_{B} is the concentration of Barquat® MB-80 (in ppm) in a Lonzabac® 12.100/Barquat® MB-80 mixture, which yielded a thousand fold reduction in the microorganism count of the process water. Q_{b} is the concentration of Barquat® MB-80 alone (in ppm) required to yield a thousand fold reduction in the microorganism count of the process water.

When the value of (Q_{A}/Qₐ+Q_{B}/Q_{b}) is less than one, the mixture is synergistic. Values for (Q_{A}/Qₐ+Q_{B}/Q_{b}) of 1 and greater than 1, represent an additive effect and an antagonistic effect, respectively.

Based on the data in Table 1, Qₐ, Q_{A}, Q_{b}, and Q_{B} are 60, 30, 50, and 20, respectively. The synergism value is, therefore, 0.9. Hence, the mixture is synergistic.

### EXAMPLE 2

The procedure in Example 1 was repeated substituting peracetic acid for Barquat® MB-80 with the solutions in Table 2 below. The results are shown below.

TABLE 2

| | | | Logarithmic Growth |
|---|---|---|---|
| | | | Reduction relative to the |
| | | Colony forming units per | Control Experiment |
| | | milliliter of sample | [log (CFU/ml of control |
| Lonzabac ® 12.100 | Peracetic Acid | (CFU/ml) after overnight | plate) − log (CFU/ml of |
| (ppm) | (ppm) | incubation | experimental plate)] |
| 15 | 0 | 6.55 × 10⁷ | 0.7 |
| 30 | 0 | 6.40 × 10⁶ | 1.7 |
| 60 | 0 | 3.25 × 10⁵ | 3.0 |
| 90 | 0 | 1.00 × 10⁵ | 3.5 |
| 0 | 20 | 1.43 × 10⁷ | 1.3 |
| 0 | 50 | 1.00 × 10⁵ | 3.2 |
| 15 | 10 | 1.86 × 10⁷ | 1.2 |
| 15 | 20 | 4.2 × 10⁶ | 1.9 |
| 30 | 10 | 5.30 × 10⁶ | 1.8 |
| 30 | 20 | 2.2 × 10⁵ | 3.2 |

| | | |
|---|---|---|
| Control | 3.16 × 10⁸ | — |

Synergism for the solutions in Table 2 was calculated as described in Example 1. Based on the data in Table 1, Qₐ, Q_{A}, Q_{b}, and Q_{B} are 60, 30, 50, and 20, respectively. The synergism value is, therefore, 0.9. Hence, the mixture is synergistic.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed:

1. A composition for inhibiting microbial growth comprising a biocidal effective amount of at least one oxidizing or non-oxidizing biocide and a triamine having the formula R—NH—(CH₂)₃—NH—(CH₂)₃—NH₂ where R is a substituted or unsubstituted C₈ to C₁₈ alkyl, C₈ to C₁₈ alkenyl, C₈ to C₁₈ alkynyl, or C₈ to C₁₈ cycloalkyl or aryl, and R is optionally interrupted with one or more heteroatoms.

2. The composition of claim 1, wherein the biocide is a non-oxidizing biocide selected from the group consisting of dibromonitrilopropionamide, decylthioethanamine (DTEA), quaternary ammonium compounds (QAC), polymeric quaternary ammonium compounds (polyquats), dodecyl guanidine hydrochloride (DGH), β-bromo-β-nitrostyrene (BNS), tetrakishydroxymethylphosphonium sulfate (THPS), bis(trichloromethyl)sulfone (BTS) and combinations thereof.

3. The composition of claim 2, wherein the non-oxidizing biocide is N-(C₁₂-C₁₆ alkyl)-N-benzyl-N,N-dimethylammonium chloride.

4. The composition of claim 1, wherein the biocide is an oxidizing biocide compound selected from the group consisting of peracetic acid, ozone, sodium hypochlorite, calcium hypochlorite, lithium hypochlorite, chlorine dioxide, bromochlorodimethylhydrantoin, dichloromethylethylhydrantoin, dichlorodimethylhydrantoin, trichlorocyanuric acid, sodium dichlorocyanuric acid, stabilized bromine, bromine chloride, brominated sulfamic acid, chlorinated sulfamic acid, sodium percarbonate, potassium monopersulfate, magnesium monoperphthalate, potassium permanganate, hydrogen peroxide, and combinations thereof.

5. The composition of claim 4, wherein the oxidizing biocide is peracetic acid.

6. The composition of claim 1,where R is an unsubstituted C₈ to C₁₈ alkyl, C₈ to C₁₈ alkenyl, C₈ to C₁₈ alkynyl, or C₈ to C₁₈ cycloalkyl or aryl.

7. The composition of claim 1, wherein the triamine is N,N-bis(3-aminopropyl)-dodecylamine or bis(3-aminopropyl)octylamine.

8. The composition of claim 1, wherein the concentration of biocide in the composition ranges from about 1 to about 500 parts per million.

9. The composition of claim 1, wherein the concentration of triamine in the composition ranges from about 1 to about 500 parts per million.

10. The composition of claim 1, wherein the weight ratio of biocide to triamine ranges from about 1:500 to about 500:1.

11. The composition of claim 10, wherein the weight ratio of biocide to triamine ranges from about 1:250 to about 250:1.

12. A method of controlling the growth of microorganisms in an aqueous solution comprising adding the composition of claim 1 to the solution.

13. The method of claim 12, wherein the aqueous solution is selected from the group consisting of water from a pulp and paper system, cooling water, food or beverage process water, and water present in an air conditioning system.

14. The method of claim 12, wherein the microorganisms are one or more organisms selected from the group consisting of bacteria, fungi, mold, and protozoa.

15. The method of claim 14, wherein the microorganisms comprise bacteria from the genus Legionella.

16. A composition comprising a synergistic microbiocidally effective amount of (a) N,N-bis(3-aminopropyl)dodecylamine and (b) peracetic acid.

17. The composition of claim 16, wherein the composition comprises from about 30 to about 1,000 ppm of N,N-bis(3-aminopropyl)dodecylamine and from about 20 to about 1,000 ppm of peracetic acid.

18. The composition of claim 17, wherein the composition comprises from about 30 to about 200 ppm of N,N-bis(3-aminopropyl)dodecylamine and from about 20 to about 200 ppm of peracetic acid.

19. A composition comprising a synergistic microbiocidally effective amount of (a) N,N-bis(3-aminopropyl)dodecylamine and (b) N-(C₁₂-C₁₆ alkyl)-N-benzyl-N,N-dimethylammonium chloride.

20. The composition of claim 19, wherein the composition comprises from about 30 to about 1,000 ppm of N,N-bis(3-aminopropyl)dodecylamine and from about 20 to about 1,000 ppm of N-(C₁₂-C₁₆ alkyl)-N-benzyl-N,N-dimethylammonium chloride.

21. The composition of claim 20, wherein the composition comprises from about 30 to about 200 ppm of N,N-bis(3-aminopropyl)dodecylamine and from about 20 to about 200 ppm of N-(C₁₂-C₁₆ alkyl)-N-benzyl-N,N-dimethylammonium chloride.

22. A method of controlling the growth of microorganisms in an aqueous solution comprising adding the composition of claim 17 to the solution.

23. A method of controlling the growth of microorganisms in an aqueous solution comprising adding the composition of claim 20 to the solution.

24. A composition comprising N,N-bis(3-aminopropyl)-dodecylamine, a peracetic acid precursor, and, optionally, a peracetic acid generator.

25. The composition of claim 24, wherein the peracetic acid precursor is tetraacetylethylene diamine.

26. The composition of claim 25, wherein the peracetic acid generator is a peracid.
